(19) United States
(12) Patent Application Publication
(10) Pub. No.: US 2010/0004873 A1
(43) Pub. Date: Jan. 7, 2010

(54) METHOD OF PROCESSING BIOLOGICAL ANALYSIS DATA AND EXPERT SYSTEM OF BIOLOGICAL ANALYSIS APPLYING THIS METHOD

(75) Inventors: Patrick RAMBAUD, Montreal (CA); Lionel Chapy, Chanonat (FR); Nicolas Roux, Boissejour (FR)

Correspondence Address: YOUNG & THOMPSON, 209 Madison Street, Suite 500, Alexandria, VA, 22314, US

(73) Assignee: IPPM HOLDING, LUXEMBOURG (LU)

(21) Appl. No.: 12/484,732

(22) Filed: Jun. 15, 2009

Related U.S. Application Data
(63) Continuation-in-part of application No. 11/397,631, filed on Apr. 5, 2006, now abandoned.
Application No. 11/397,631 is a continuation-in-part of application No. 10/111,900, filed on Sep. 25, 2002, now abandoned.

(30) Foreign Application Priority Data
Jan. 19, 2001 (FR) 01 00771

(51) Int. Cl. G06F 19/00; G01N 33/48

(52) U.S. Cl. 702/19

(57) ABSTRACT

An expert system of biological analysis includes a collecting engine to collect and represent data resulting from biological measurements carried out on a human or animal subject and defining a biological profile, and personal data relating to the human or animal subject.

### CROSS REFERENCE TO RELATED APPLICATIONS

This application is a Continuation-in-Part of copending application Ser. No. 11/397,631 filed on Apr. 5, 2006; which is a Continuation-in-Part of application Ser. No. 10/111,900 filed on Sep. 25, 2002; which is the 35 U.S.C. 371 national stage of international application PCT/FR02/00218 filed on Jan. 18, 2002, which claimed priority of French application No.: 01 00771 filed on Jan. 19, 2001. The entire contents of each of the above-identified applications are hereby incorporated b reference.

### FIELD OF THE INVENTION

The present invention relates to an expert system of biological analysis.

In the field of biological analysis, there are already methods of determining the biological profile of a person from a set of measurements of characteristic physiological parameters. Starting from these biological profiles and data relating to the person concerned such as his age, his sex, his physical condition, the practitioner can then produce a set of conclusions or outputs leading to a diagnosis. A biological profile is comprised in practice of a set of specific profiles, such as a protein profile or lymphocyte typing.

The increase in the number of parameters involved in the determination of a biological profile makes it more and more difficult to establish consistent conclusions or outputs. In order to satisfy the expectations of practitioners who prescribe biological analyses, expert systems of biological analysis have been developed. These expert systems procure for users the processing of a set of items corresponding to biological measurements data and to personal data, and provide conclusions which can be used directly by the prescribing practitioner.

The methods of processing biological data that are used in these expert systems require a set of rules each applied to a determined combination of items among a global set of items corresponding to a set of measurements, examinations, dosages carried out on a patient or personal data. These rules lead to a set of conclusions which are drafted beforehand by one or more expert practitioners.

It has been shown in practice that the number of possible theoretical conclusions in an expert system of biological analysis, intended to integrate as complete as possible a biological profile in the current state of the techniques available in biological analysis, is so high that the feasibility of such an expert system and its implementation on conventional data-processing equipment other than large-capacity calculation and storage machines could be implicated.

### BACKGROUND OF THE INVENTION

Buchanon et al. (WO 00/42487) disclose a computer-implemented interactive expert system and method of using it for real time decision support in the medical field. The expert system disclosed in Buchanon et al. contains rules that can be created and modified. Pertinent subsets of rules can be selected to reduce response times by limiting the number of conclusions. However, selecting subsets of rules in order to limit the number of conclusions, may on the one hand be very time wasting and on the second affect the accuracy and rigour of the conclusions supplied to the user

### SUMMARY OF THE INVENTION

The aim of the present invention is to propose an expert system of processing biological analysis data which on the one hand resolves effectively the question of the volume of data to be processed and consequently render such an expert system realisable, and which on the other hand procures for the practitioner using it a better relevance of the conclusions for interpretation of the analysis results.

This aim is achieved with an expert system of biological analysis, comprising:
a collecting engine to collect and represent, in the form of a set of groups of items, data resulting from biological measurements carried out on a human or animal subject and defining a biological profile, and personal data relating to the said human or animal subject,
a set of several groups of pre-established rules;
an inference engine to issue a set of conclusions by applying at least one group of the said set of groups of rules to:
   at least one group of items selected from the said set of groups of items; and/or
   at least one former set of conclusions issued by said inference engine,
      wherein it also comprises a gathering engine to minimize the number of conclusions in the said set of conclusions issued by the said inference engine.

Such gathering engine accomplishing gathering operations have the effect of making possible the implementation of an expert system of biological analysis on a personal office-based or portable data-processing apparatus, without affecting the accuracy and rigour of the conclusions supplied to the user.

It is to be noted that in the present invention gathering operations relates to gathering the number of conclusions in a set of conclusions and not to the fusion of chained rules as taught in the pending U.S. Pat. No. 5,442,792 which discloses a compiling method for an expert system.

It is also to be noted that an “engine” can be a computer program, one or more software run by a computer or made of computer executable instructions run by a computer, a processor or the like. Many structures of “engine” are well known in the expert systems known in the art, such as those described in the U.S. Pat. No. 5,263,126.

It is still to be noted that a “conclusion” means the output of a rule executed by an engine and more particularly by an inference engine. A “conclusion” can, for example, be a numerical value, a word, a sentence or the like, and is well known in the art.

Another aim of the method of data processing according to the invention is to permit the realisation of an expert system of biological analysis which integrates genetic profile data, knowledge of which is henceforth regarded as essential for the diagnosis and treatment of an increasing number of affections and pathologies.

This aim is achieved by an expert system comprising a genetic data collecting engine collecting data relating to the genetic profile of the said human or animal subject in the form of genetic items, said genetic data collecting engine also comprising a set of rules for the interpretation of the said genetic data.

The genetic data collecting engine can also comprise a processing of genetic data relating to this patient in the form of a set of genetic items associated with a set of genes studied in this patient, this processing comprising the application of rules of genetic interpretation applied at the same time to biological items and to genetic items.

With this expert system combining interpretation of a biological profile and interpretation of a genetic profile, it becomes possible to propose more accurate and more relevant conclusions due to the taking into account of affections linked with the genes.

It is to be noted that document WO 01/16860 discloses artificial intelligence system for a genetic analysis, which does not involve a gathering operation of a set of conclusions that have resulted from a group of rules, as proposed by the method according to the invention.

Said collecting engine, said set of several groups of pre-established rules, said inference engine and said gathering engine can also be integrated into an automated system including operations determining a biological and a genetic profile of a patient.

The expert system according to the present invention can further comprise a coupled knowledge base, so that it can reach all the information present in the knowledge base. Thus, with the expert system according to the present invention the interpretation of a biological profile and interpretation of a genetic profile, it becomes possible to propose more accurate and more relevant conclusions due to the taking into account of information present I the coupled knowledge base.

The biological profile taken into account in the expert system according to the present invention also includes a protein profile.

The biological profile taken into account in the expert system according to the present invention also includes a lymphocyte typing.

According to another aspect of the invention it is proposed an expert system of biological analysis, comprising computer executable instructions defining:
a collecting engine to collect and represent, in the form of a set of groups of items, data resulting from biological measurements carried out on a human or animal subject and defining a biological profile, and personal data relating to the said human or animal subject,
a set of several groups of pre-established rules;
an inference engine to issue a set of conclusions by applying at least one group of the said set of groups of rules to:
   at least one group of items selected from the said set of groups of items; and/or
   at least one former set of conclusions issued by said inference engine,
      wherein it also comprises a gathering engine to minimize the number of conclusions in the said set of conclusions issued by the said inference engine.

According to another aspect of the invention it is proposed a method for processing biological analysis data, comprising:
collecting and representing, in the form of a set of groups of items, data resulting from biological measurements carried out on a human or animal subject and defining a biological profile, and personal data relating to the said human or animal subject,
issuing a set of conclusions by applying at least one group of a set of several groups of pre-established rules to:
   at least one group of items selected from the said set of groups of items; and/or
   at least one former set of conclusions issued by said inference engine,
      wherein it also comprises gathering at least two conclusions from the said set of conclusions to minimize the number of conclusions in the said set of conclusions.

### BRIEF DESCRIPTION OF THE DRAWINGS

Other advantages and characteristics of the invention will appear upon examination of the detailed description of an embodiment, which is no way limitative, and of the attached drawings in which:

FIG. 1 is a functional diagram of an expert system of biological analysis according to the invention,

FIG. 2A illustrates an example of internal structure of the engine of an expert system according to the invention, relating more particularly to rules of the inflammatory reaction, and

FIG. 2B illustrates an example of internal structure of the engine of an expert system according to the invention, relating more particularly to rules of interpretation of immunoglobulin.

### DETAILED DESCRIPTION OF THE INVENTION

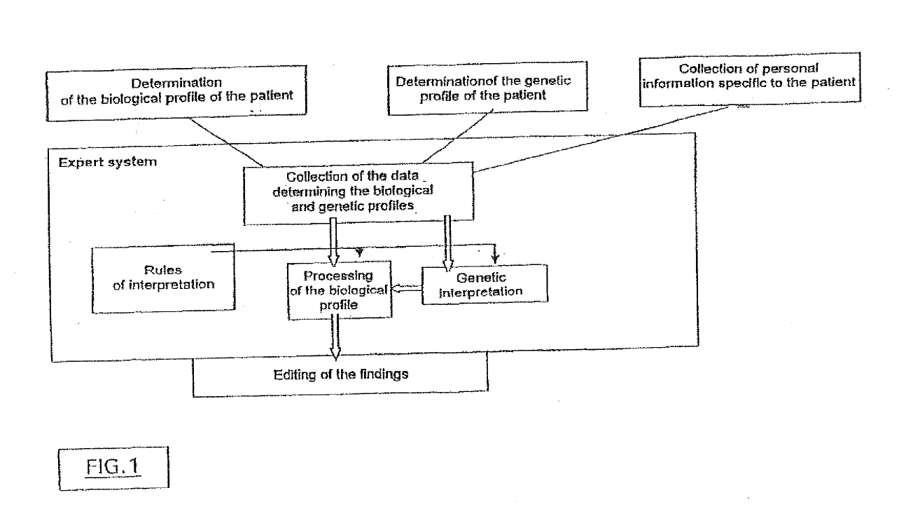

An expert system according to the invention of biological analysis according to the invention can in practice be implemented within a computer such as an office computer or a portable computer, and accessed locally or remotely. Its internal architecture, which can conform to current standards applying to expert systems, includes, with reference to FIG. 1, a module for collecting data determining profiles, respectively biological (protein in particular) and genetic profiles, of a patient, a module for collecting personal information specific to the patient, rules of interpretation applied to a processing of the biological profile realised with a genetic interpretation, and an editing of conclusions or outputs that can be used by a practitioner user.

The set of rules contained in this expert system according to the invention is organised into groups of rules each group corresponding to a group of specific analysis among several groups of analysis. For example, one may consider the group of rules of the inflammatory reaction or the group of rules interpreting immunoglobulins.

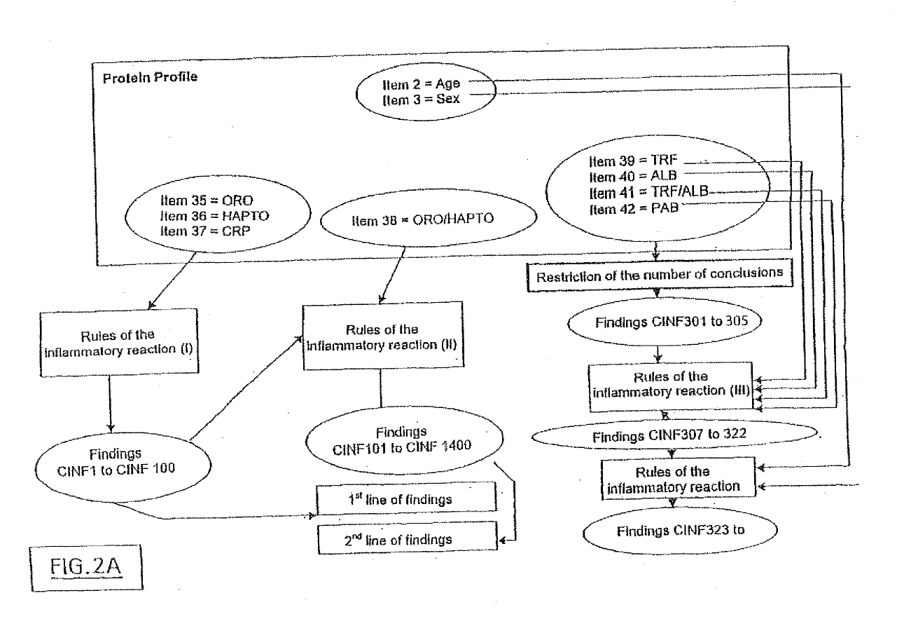

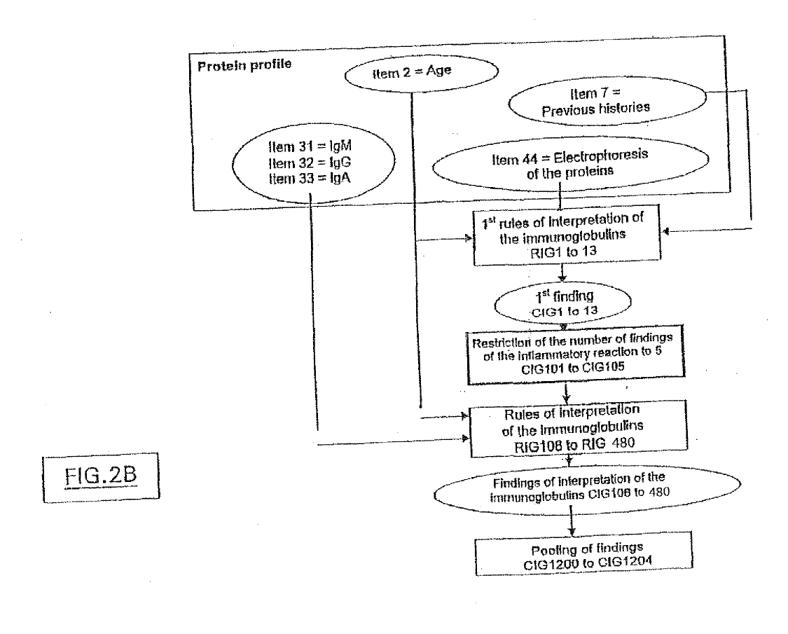

An embodiment of an expert system according to the invention will now be described, with reference to FIGS. 2A and 2B, being limited, for reasons of fullness of the description and clarity, only to the protein profile of a patient, it being understood that other specific biological profiles could be processed in an equivalent manner within the scope of the present invention.

In this expert system, a protein profile comprises optional items such as Item 43=ROP or Item 45=C4 and a set of obligatory items such as the following items:

Item 2 = Age
Item 3 = Sex
Item 35 = ORO
35.1 = Normal
35.2 = Increased
35.3 = Much increased
35.4 = Reduced
Item 36 = HAPTO
36.1 = Normal
36.2 = Increased
36.3 = Much increased
36.4 = Reduced
36.5 = Much reduced
Item 37 = CRP
37.1 = Normal < 33%
37.2 = Normal increased
37.3 = Increased
37.4 = Much increased
37.5 = Very much increased . . .
Item 39 = TRF
39.1 = Normal
39.2 = Increased (obligatorily > 119, not below)
39.3 = Reduced
Item 40 = ALB
40.1 = Normal or increased (>89%)
40.2 = Reduced (<89%)
Item 41 = TRF/ALB
41.1 = Normal
41.2 = Increased
Item 42 = PAB
42.1 = Normal or increased (>84%)
42.2 = Reduced (<84%)
Item 44 = Electrophoresis of the proteins effected
NO
IgM
IgG
IgA
Monoclonal peak not M, not G, not A
Double monoclonal peak
Absence of monoclonal protein

The expert system according to the invention includes rules of the inflammatory reaction such as the following rules:
RINF1=35.1+36.1+37.1=CINF1
RINF2=35.1+36.1+37.2=CINF2
. . .
RINF100=35.4+36.5+37.5=CINF100
   The conclusions associated with these rules of the inflammatory reaction are for example written up in the following way:
CINF1=No inflammatory reaction as the proteins of the inflammation (CRP, Alpha-1-glycoprotein or orosomucoid, haptoglobin) are normal.
CINF2=The proteins of the inflammatory reaction are all in normal values. However, the level of CRP, although normal, may suggest the presence of microinflammations (to be taken into consideration in the assessment of the cardiovascular risk after formal elimination of any other potentially phlogogenic spot).
CINF100=The results lean towards an inflammatory process based solely on the strong increase in the CRP. This may be the start of an inflammatory process since the CRP, a protein of acute inflammation, increases more quickly than the other proteins of the inflammation. Such an induction leans towards an infectious and/or inflammatory spot that is at the present time recent and very active, kept in an active state or in re-induction phase. The low level of haptoglobin is favourable for a hemolysis. Any haptoglobin result below 50% can be considered pathological. It may thus be of benefit to seek the cause of this hemolysis. Here, the reduced level of alpha-1-glycoprotein need not suggest a medicament treatment in the first place, but rather a protein leak, or a hepatocytic insufficiency.

Some of the conclusions CINF1 to CINF100 are impossible. There are 60 different conclusions left. Although there are actually only 60 different conclusions, this would lead to far too great a number of rules and conclusions. It is thus proposed to gather these 60 conclusions to obtain 6 new conclusions as represented in FIG. 2A. This gathering operation is made by grouping the conclusions by meaning. The new conclusions are built in the following way:

### RINF301=CINF1 or CINF2 or CINF3 or CINF6 or . . . or CINF83=CINF301

The 6 new conclusions are:
1) CINF301: no, or very slight, inflammatory reaction
2) CINF302: inflammatory reaction based solely on the increase in CRP
3) CINF303: inflammatory reaction with increase in only one protein of the chronic reaction
4) CINF304: clear inflammatory reaction with normal CRP
5) CINF305: clear inflammatory reaction with increased CRP
6) CINF306: reduction in the proteins of the chronic inflammation
   If 6 conclusions CINF301 to CINF306 are considered, linked with items 39 (TRF), 40 (ALB), 41 (TRF/ALB), 42 (PAB), 6×3×2×2×2, i.e. 144 gathering rules must be provided.
   The 144 possible different rules include:
RINF307=CINF301+39.1+40.1+41.1+42.1=CINF307
. . .
RINF450=CINF306+39.3+40.2+41.2+42.2=CINF450
   144 different conclusions are obtained. A gathering step is then performed to link the 144 conclusions obtained. The conclusions CINF307 to CINF450 are linked together to obtain a reduced number of conclusions.

The interpretation of the Ig (immunoglobulins) in the protein profile will now be considered. The items concerned are 31 (IgM), 32 (IgG) and 33 (IgA). The interpretation of the inflammatory conclusions CINF1 to CINF100 is different. There be a different gathering of the inflammatory conclusions CINF1 to CINF100 to obtain 5 new conclusions. These 5 new conclusions will be then linked to the items 31 to 33.

The interpretation of the Ig (immunoglobulins) in the protein profile is different depending on whether there is or not a monoclonal protein. Now, the presence of a monoclonal protein is not visible in the protein profile but in another analysis which is electrophoresis of the proteins. When a monoclonal protein is found, the interpretation stops there, and this finding is not linked with an inflammatory reaction. Thus, the interpretation of the Ig starts with the processing of item 44 “Electrophoresis of the proteins”.

The reply may be:
44.1: no (1^{st} finding),
44.2: yes with presence of a monoclonal protein (new 1^{st} finding),
44.3: yes with absence of monoclonal protein (new 1^{st} finding),
   The first finding can be established in the following way:
RIG1=44.2+12.1=CIG1
. . .
RIG13=17.2=CIG13
CIG1=The electrophoresis and the immunoelectrophoresis revealed a monoclonal IgM. Given the patient's age, one must think first of a sub-acute or chronic severe infection or viral or bacterial origin. This suggests an associated immunodeficiency.
CIG13=The values of the Ig reflect all of the defences acquired during life as a function of encounters with the different pathogens. At adult age, in a healthy person, this level does not vary much. It is thus perfectly possible that a level outside the normal values has no pathological connotation, but be a perfectly physiological level for the patient. What is interesting is the assessment of the variation over two samples several months apart. Not having any prior history for this patient, the different etiologies proposed enjoy only indicative status, as the interpretation must be carried out above all in relation to the clinical context.

Any individual can present Ig levels outside the standard values without this being pathological. What is pathological is the variation in this level of Ig over two taking, hence the processing of item 7 “previous histories”. If the reply is no, this means a 2^{nd} finding of a general order before the actual processing of the Ig.

Items 31, 32, 33 must then be linked with the inflammatory reaction. As précised above, we create 5 new inflammatory conclusions which summarize inflammatory reactions by meaning in the immunoglobulin interpretation context. These conclusions are:
CIG101: no inflammatory reaction
CIG102: slight inflammatory reaction
CIG103: inflammatory reaction due solely to CRP
CIG104: inflammatory reaction present (1, 2 or 3 proteins)
CIG105: reduction of the proteins of the inflammatory reaction.
   The 3^{rd} finding will thus be chosen from among the following rules:
RIG101=CINF1 or CINF18 or . . . or CINF78=CIG101

The new inflammatory conclusions CIG101 to CIG105 are obtained by a different gathering of the conclusions CINF to CINF100.

We now combine items such as I31×I32×I33 with the new 5 inflammatory conclusions (CIG101 to CIG105) and we obtain: 5×3×5×5=375 new rules.

The 375 rules for the 3^{rd} finding are then established, such as by way of example:
RIG106=CIG101+31.1+32.1+33.1=CIG106
. . .
RIG480=CIG105+31.5+32.3+33.5=CIG480
. . .

By combining items I31 to I33 with the new 5 inflammatory conclusions we obtain 375 conclusions CIG106 to CIG480

Complementary rules as a function of age are added to take account of the situations where each time there will be an inflammatory reaction (CIG104) without any increase in the Ig, or with a reduction in the IgM. In order to create these complementary rules, a gathering of certain of the CIGxxx conclusions mentioned above is carried out in order to end up with 5 conclusions CIG1200, CIG1201, CIG1202, CIG1203, CIG1204 which are used in the establishment of these complementary rules.
RIG1200=CIG152 or CIG153 or . . . or CIG180=CIG1200
RIG1205=CIG1200+750.2=CIG1205

Moreover, many conclusions CIG106 to CIG480 have a similar text, only some words are different depending on the rule input. For example we have these 2 rules:
RIG167=CIG102+31.4+32.2+33.3+MOL.746 (item for drug intake Corticoid therapy)=CIG167
RIG168=CIG102+31.4+32.2+33.3+MOL.753 (item for drug intake Methotrexate)=CIG168
   And the corresponding conclusions are:
CIG167=“In this patient case, use of corticoid therapy may explain such immunoglobulin values.”
CIG168=“In this patient case, use of methotrexat may explain such immunoglobulin values.”

In order to avoid writing 2 rules and 2 conclusions we create only one rule and conclusion use the principle of “a magic hole”:
RIG167N=CIG102+31.4+32.2+33.3+(MOL.746 or MOL.753)=CIG167N

The rule combines the conclusion CIG102, items 31, 32, 33 and one of the drugs among MOL.746 and MOL.753.

We then create the following conclusion:
CIG167N=“In this patient case, use of [[MOL]] may explain such immunoglobulin values.”

When the algorithm will find the specific characters “[[MOL]]” (which we named “the magic hole”), it will replace it by a human readable text of the item which activates that rule. If the item MOL.746 is selected in the patient profile, the rule will be activated and the content of the conclusion will be replaced by “In this patient case, use of corticoid therapy may explain such immunoglobulin values.”

An embodiment of the method of processing data according to the invention will now be described, for a combined interpretation of the genetic profile and cardiovascular risk.

Firstly, a non-exhaustive list of genes that can be interpreted within the scope of the expert system of biological analysis according to the invention is provided in table I below. For each gene, a + symbol in a column indicates that this gene plays a part in the characteristic corresponding to this column, and conversely a − symbol in another column indicates that the same gene does not play a part in the characteristic corresponding to this other column. Thus, by way of example, the gene CYP1A1 plays a part in the case of smoker and as regards nutrigenetics, but not as regards pharmacogenetics, immunogenetics and for oxidative stress. Thus, each + symbol in this table corresponds to links and rules which must be written and integrated into the expert system.

There follow, by way of non-limitative example, extracts of biological interpretation supplied by an expert system according to the invention, regarding cardiovascular risk:

“in the light of the biological results, there is no atherogenic risk. The other risk factors must therefore be explored, as nearly 20% of patients who have cardiovascular problems present a normal or sub-normal biology.”
[ . . . ]
“Hyperhomocystinemia caused by congenital deficiency of the enzymes involved in its biosynthesis is much more rare. For example, cystathionine-beta-synthase deficiency is estimated at 1/20000 subjects who, in addition to cardiovascular risk, also have mental backwardness, and a dislocation of the crystalline lens, osseous deformations. On the other hand, 5-10 methylinetetrahydrofolate reductase deficiency is more frequent, being estimated at 5% of the general population, and is the major cause of genetic predisposition to moderate hyperhomocystinemia. These patients often present cardiovascular disorders in the first years of life [ . . . ]”
This constitutes an indication for conducting genetic tests in order to know whether the increase in homocysteine is genetic in origin or not.
“Although the E2 allele seems to play a part in type III hyperlipoproteinemias, the E4 allele is also more involved in cardiovascular diseases. The E2/E4 genotype, although infrequent, thus substantially increases the risks of cardiovascular problems. Generally speaking, the average cholesterolemia of E4/E3 subjects is greater than that of E3/E3 subjects, which is itself greater than that of E3/E2 subjects. In the same way, the average concentration of LDL cholesterol in E4/E3 subjects is greater than that of E3/E3 subjects, which is itself greater than that of E2/E2 subjects. On the other hand, the triglycerides are significantly higher in E2/E2; E3/E2; E4/E2 subjects than in E3/E3; E4/E3 subjects.”

This finding reflects a direct relationship between interpretation of a genetic profile and interpretation of a biological profile (cholesterol, triglycerides).

There is presented below an example of a finding reflecting a direct relationship between genetics and diet:

“Subjects carrying the E4 allele are more sensitive to hypolipemic and hypocholesterolemic diets. In the same subjects, the return to a diet rich in fats, in particular in saturated fatty acids, leads to a greater increase in plasmatic cholesterol.”

The expert system according to the invention can also take account, in the conclusions supplied to the user, of a direct relationship between the interpretation of the genetic profile and data relating to the medicament treatment that are obtained from personal information specific to the patient, as illustrated by the finding presented below:

“Subjects carrying the E2 allele and affected by hyperlipoproteinemia of lib type respond well to treatment by gemfibrozil and by statins (simvastatin and lovastatin). Among subjects affected by hyperlipoproteinemia of lia type, carriers of the E2 or E3 allele respond well to treatment by statins. Subjects carrying the E4 allele would on the other hand respond less well to hypolipidemic medicamentous treatments, with the exception, perhaps, of probucol.”

The invention is, of course, not limited to the examples which have just been described and numerous modifications can be made to these examples without exceeding the scope of the invention. In particular, provision can be made for complete automation of the operations for determining the biological profile and the genetic profile of a patient, and the combined treatment of these profiles. Moreover, it will easily be understood that an expert system of biological analysis according to the invention can also be coupled with databases and knowledge bases. In addition, within the framework of the present invention, the biological profile not only includes several families of determinations and biological analysis which are henceforth well established such as protein profiling or lymphocyte typing, but also other profiles in the process of being developed or which will be proposed in the future. In the same way, the expert system according to the invention is intended to take account of increasingly complex genetic profiles as scientific and technological advances occur in this field.

TABLE I

| | Predisposition | | | | | |
|---|---|---|---|---|---|---|
| | to | | | | | |
| Genes | disease | Pharmacogenetic | Immunogenetic | Smoker | Stress O. | Nutrigenetic |
| Phase I of | | | | | | |
| bio- | | | | | | |
| transformation | | | | | | |
| CYP1A1 | + | − | − | + | − | + |
| CYP1A2 | + | + | − | + | − | − |
| CYP2A6 | + | + | − | + | − | − |
| CYP3A4 | + | + | − | − | − | + |
| CYP2B6 | + | + | − | − | − | + |
| CYP1B1 | + | + | − | − | − | + |
| CYP2D6 | + | + | − | − | − | − |
| CYP2E1 | + | − | − | − | − | + |
| CYP2C19 | + | + | − | − | − | − |
| CYP2C9 | − | + | − | − | − | − |
| MEH | + | − | − | + | − | − |
| ALDH | + | − | − | + | − | − |
| ADH2 | + | − | − | + | − | − |
| Phase II of | | | | | | |
| bio- | | | | | | |
| transformation | | | | | | |
| GSTM1 | + | + | + | + | + | + |
| GSTM3 | + | − | − | − | + | + |
| GSTT1 | + | − | − | + | + | + |
| GSTP1 | + | + | − | − | + | + |
| NAT2 | + | + | + | + | − | + |
| NAT1 | + | + | + | + | − | + |
| Trigger | | | | | | |
| genes | | | | | | |
| Osteoporosis | | | | | | |
| Vit D3 | + | − | − | − | − | − |
| Col1A1 | + | − | − | − | − | − |
| ER | + | − | − | − | − | − |
| CTR | + | − | − | − | − | − |
| AIDS | | | | | | |
| CCR5 | + | − | − | − | − | − |
| SDF1 | + | − | − | − | − | − |
| CCR2 | + | − | − | − | − | − |
| CXCR4 | + | − | − | − | − | − |
| Breast | | | | | | |
| cancer | | | | | | |
| BRCA1 | + | − | − | − | − | − |
| BRCA2 | + | − | − | − | − | − |
| Prostate | | | | | | |
| cancer | | | | | | |
| AR | + | − | − | − | − | − |
| Hereditary | | | | | | |
| trombophilia | | | | | | |
| Factor V | + | − | − | − | − | − |
| Hemochromatosis | | | | | | |
| HFE | + | − | − | − | − | − |
| Bronchial | | | | | | |
| and allergic | | | | | | |
| asthma | | | | | | |
| CC16 | + | − | − | − | − | − |
| AAT-locus | + | − | − | + | − | − |
| HNMT | + | − | − | + | − | − |
| PAFAH | + | − | − | + | − | − |
| AACT | + | − | − | + | − | − |
| Primary | | | | | | |
| Hypercholesteremia | | | | | | |
| LDLR | + | − | − | − | − | − |
| APOB | + | − | − | − | − | − |
| Cardiovascular | | | | | | |
| risk | | | | | | |
| MTHFR | + | − | − | − | − | − |
| ACE | + | − | − | − | − | − |
| Efflux genes | | | | | | |
| MDR1 | − | + | − | − | − | − |
| MDR3 | − | + | − | − | − | − |
| LRP | − | + | − | − | − | − |
| MRP1 | − | + | − | − | − | − |
| Other | | | | | | |
| metabolizing | | | | | | |
| genes | | | | | | |
| NQO1 | + | − | − | − | + | − |
| Cytokine | | | | | | |
| genes | | | | | | |
| IL-1a | + | − | + | − | − | − |
| IL-1b | + | − | + | − | − | − |
| ILRN | + | − | + | − | − | − |
| IL-2 | + | − | + | − | − | − |
| IL-4 | + | − | + | − | − | − |
| IL-6 | + | − | + | − | − | − |
| IL-9 | + | − | + | − | − | −− |

We claim:

1. An expert system for processing biological analysis data, comprising:
a processor which executes:
a collecting engine to
i) collect measurement data resulting from biological measurements carried out on a human or animal subject, the collected data defining a biological profile,
ii) collect personal data relating to the human or animal subject, and
iii) represent the measurement data and the personal data in the form of a set of groups of data items, the set of groups of data items comprising at least a first data item group, a second data item group, and a final data item group;
a set of groups of pre-established rules, the set of groups of pre-established rules comprising at least a first rule group, a second rule group, and a final rule group, each rule group corresponding to a different group of specific analysis,
application of the first rule group to the first data item group resulting in a first group of conclusions of interpretation;
a gathering engine to gather the first group of conclusions of interpretation and output a restricted group of conclusions of interpretation, the restricted group of conclusions of interpretation having a reduced the number of conclusions of interpretation as compared to the first group of conclusion of interpretation,
application of i) the restricted group of conclusions of interpretation and ii) the second data item group to the second rule group resulting in a second group of conclusions of interpretation,
application of i) the second group of conclusions of interpretation and ii) the final data item group to the final rule group resulting in a set of diagnostic conclusions; and
an inference engine which comprises the gathering engine and applies each group of rules to at least one of
i) a corresponding one of the data item groups and
ii) the restricted group of conclusions of interpretation,
the inference engine displaying the set of diagnostic conclusions as an output usable by a practitioner user of said expert system.

2. The expert system according to claim 1, further comprising:
a genetic data collecting engine collecting data relating to a genetic profile of the human or animal subject in the form of genetic data items, said genetic data collecting engine also comprising a set of rules for the interpretation of the genetic data items.

3. The expert system according to claim 1, wherein said collecting engine, said set of groups of pre-established rules, said inference engine and said gathering engine are integrated into an automated system including operations to determine a biological profile of the subject and a genetic profile of the subject.

4. The expert system according to claim 1, further comprising a coupled knowledge base.

5. The expert system according to claim 1, wherein the biological profile includes a protein profile.

6. The expert system according to claim 1, wherein the biological profile includes a lymphocyte typing.

7. The expert system according to claim 1, wherein the gathering engine reduces the number of conclusion of interpretation so that the restricted group of conclusions of interpretation has a fact of twn reduced the number of conclusions of interpretation as compared to the first group of conclusion of interpretation.

8. The expert system of biological analysis according to claim 1, wherein the total quantity of conclusions in the restricted group of conclusions of interpretation is approximately ten times fewer than the total quantity of conclusions in the first group of conclusions of interpretation.

9. An expert system of biological analysis, comprising:
a collecting engine that collects i) biological data resulting from biological measurements carried out on a human or animal subject, the biological data defining a biological profile of the subject, and ii) personal data relating to the subject, the collected biological data and the collected personal data organized into a set of groups of data items, the set of groups of data items comprising at least a first data item group and a final data item group;
a set of groups of pre-established rules, the groups of pre-established rules comprising at least a first rule group which results in a first group of conclusion of interpretations, and a final rule group which results in a set of diagnostic conclusions displayed to a practitioner user of said expert system, each rule group corresponding to a different group of specific analysis;
a gathering engine that gathers the first group of conclusions of interpretation and outputs a restricted group of conclusions of interpretation, the restricted group of conclusions of interpretation having a reduced the number of conclusions of interpretation as compared to the first group of conclusion of interpretation; and
an inference engine, comprising the gathering engine, that applies the final rule group to the final data item group for resulting in the set of diagnostic conclusions displayed to the practitioner user.

10. The expert system of biological analysis of claim 9, wherein,
the groups of pre-established rules further comprises a second rule group which results in a further group of conclusion of interpretations,
the inference engine applies the second rule group to the restricted group of conclusions of interpretations for resulting in a further group of conclusions of interpretations, and
the inference engine applies the final rule group to the final data item group and to the further group of conclusions of interpretations for resulting in the set of diagnostic conclusions.

11. A method for processing biological analysis data, comprising the steps of:
collecting biological measurements data of a subject, the collected data defining a biological profile of a subject;
collecting personal data relating to the subject;
representing the collected biological measurements data and the personal data in the form of a set of groups of data items comprised of a first group of data items and a second group of data items;
issuing a first set of diagnostic conclusions based on a first group of pre-established rules being applied to the first group of data items;
gathering the first set of diagnostic conclusions to produce a reduced set of conclusions, the reduced set of conclusions having fewer conclusions than in the first set of conclusions; and
issuing a set of diagnostic conclusions based on a second group of pre-established rules being applied to the reduced set of conclusions, the diagnostic conclusions being displayed to a user,
wherein said subject is one of a human subject and an animal subject.
